(19) United States
(12) Patent Application Publication
(10) Pub. No.: US 2020/0138314 A1
(43) Pub. Date: May 7, 2020

(54) Modular ECG recording system suitable for wearable and handheld measurements

(71) Applicant: VivaLnk, Inc., Campbell, CA (US)

(72) Inventors: Nishant Doctor, Mountain view, CA (US); Jiang Li, Cupertino, CA (US)

(21) Appl. No.: 16/182,714

(22) Filed: Nov. 7, 2018

(51) Int. Cl. A61B 5/0404; A61B 5/0408; A61B 5/00; A61B 5/04; A61B 5/0432

(52) U.S. Cl. CPC A61B 5/0404; A61B 5/04087; A61B 5/0006; A61B 5/002; A61B 2562/0209; A61B 5/0432; A61B 5/04085; A61B 2560/0214; A61B 2560/0443; A61B 5/04012

(57) ABSTRACT

A modular electrocardiogram recording system includes a control patch, a wearable patch configured to be electrically connected to the control patch and to be worn on a user's skin, a handheld panel configured to be electrically connected to the control patch and to be hand held by the user. The wearable patch includes one or more sensing electrodes configured to sense a first ECG signal from the user. The handheld panel includes one or more conductive pads configured to sense a second ECG signal from the user,

### BACKGROUND OF THE INVENTION

The present application relates to wearable and portable electronic devices, and in particular, to technologies that can record electrocardiogram (ECG) signals in a flexible manner according to compatible with user's lifestyle and measurement needs.

Cardiac diseases especially arrhythmias can be detected using ECG. However, most conditions are not sustaining and cannot be captured easily by a standard 10 sec ECG. In addition, the problematic episodes are often short lasting and have already disappeared before the patient starts to feel the symptoms. Some patients may even be asymptomatic. Therefore, ambulatory ECG recordings are useful for screening, monitoring, or drug titration for cardiac patients and patients with risks or associated symptoms.

Holter ECG is the standard option for ECG measurements at home, but it is inconvenient and cumbersome with wires and the recording box. Recordings are sent to the doctor for offline analysis after the monitoring period is over. There is no prompt data sharing or analysis. The patient cannot get prompt feedback or guidance for action. Most of Holter systems can conduct 24 hours to 72 hours of monitoring, which is often not long enough for diagnosis.

There is therefore a need for improved ECG recording technologies that can conduct ECG recordings in a flexible manner compatible with patient's lifestyle and measurement needs.

### SUMMARY OF THE INVENTION

The presently application discloses a modular ECG recording systems and configurable ECG recording methods that can conduct ECG recordings in both wearable and handheld configurations. The modular ECG recording systems and configurable ECG recording methods can provide more relevant and more ECG measurements and overcomes limitations of the conventional techniques.

The disclosed modular ECG recording systems can be configured to provide long-term ECG measurements, which increase the chances for cardiac conditions to be captured. The long-term ECG measurement is performed by a wearable configuration that is comfortable to wear without wire connections to heavy equipment. The measurements can be performed while a person fulfills his or her normal daily activities. Moreover, a wearable patch in the wearable configuration can be disposable, while a control patch can be re-used for a long time, which reduces the cost and complexity of the wearable configuration.

The disclosed modular ECG recording systems can also be configured to enable handheld measurement at high signal-to-noise ratio for a focused time period. Importantly, a patient's ECG recording data for both wearable and handheld measurement configurations are analyzed together for the most accurate understanding of the patient's cardiac conditions.

In one general aspect, the present invention relates to a modular electrocardiogram recording system that includes a control patch comprising: a control system, a wearable electrical interface and a handheld interface; a wearable patch configured to be electrically connected to the control patch and to be worn on a user's skin. The wearable patch includes a flexible substrate; one or more sensing electrodes configured to be in contact with the user's skin to sense a first ECG signal; and first interface electrodes in electrical communications with the one or more sensing electrodes. The first interface electrodes can electrically connect to the wearable electrical interface in the control patch and send the first ECG signal to the wearable electrical interface. The modular electrocardiogram recording system also a handheld panel that can be electrically connected to the control patch and hand held by the user. The handheld panel includes one or more conductive pads configured to be held by and to be in contact with a user's fingers; and second interface electrodes in electrical communications with the one or more conductive pads. The one or more conductive pads can sense a second ECG signal from the user. The second interface electrodes can electrically connect to the handheld electrical interface in the control patch and send the second ECG signal to the handheld electrical interface.

Implementations of the system may include one or more of the following. The wearable patch can include a first port configured to receive the control patch, which causes the first interface electrodes to electrically connect to the wearable electrical interface in the control patch. The first port can include a bracket or a recess configured to secure the control patch to the wearable patch. The wearable patch includes an adhesive material to enable the wearable patch to adhere to the user's skin, which can attach the sensing electrodes to be in contact with the user's skin. The handheld panel can include a second port that can receive the control patch, which causes the second interface electrodes to electrically connect to the handheld electrical interface in the control patch. The second port can include a bracket or a recess configured to secure the control patch to the handheld panel. The control patch can include a signal sensing and data acquisition unit in communication with the wearable electrical interface and the handheld interface, wherein the signal sensing and data acquisition unit can receive the first ECG signal via the wearable electrical interface and configured to receive the second ECG signal via the handheld electrical interface. The control system further can include a wireless communication unit configured to transmit ECG data to an external device, the ECG data being based on at least one of the first ECG signal and the second ECG signal. The control system further can include a controller/processor configured to analyze the first ECG signal and the second ECG signal to produce the ECG data. The one or more sensing electrodes in the wearable patch can sense the first ECG signal in the user's body under the control of the controller/processor. The one or more conductive pads in the handheld panel can sense the second ECG signal in the user's body under the control of the controller/processor. The control system can further include rechargeable batteries and a charging unit, wherein the charging unit is configured to charge the rechargeable batteries when the control patch is mounted on the handheld panel, wherein the handheld panel is configured to receive power from an external source or a back-up battery. One of the one or more sensing electrodes can be used as grounding.

In another aspect, the present invention relates to a method for configurable electrocardiogram recordings. The method includes mounting a control patch to a wearable patch to be worn on a user's skin, wherein the control patch includes a control system, a wearable electrical interface, and a handheld interface, wherein the wearable patch includes a flexible substrate, one or more sensing electrodes, and first interface electrodes in electrical communications with the one or more sensing electrodes; electrically connecting the first interface electrodes to the wearable electrical interface in the control patch; sensing a first ECG signal from the user when the wearable patch is in contact with the user's skin; sending the first ECG signal to the wearable electrical interface in the control patch; mounting the control patch to a handheld panel comprising one or more conductive pads configured to be held by a user's fingers and second interface electrodes in electrical communications with the one or more conductive pads; electrically connecting the second interface electrodes to the handheld electrical interface in the control patch; sensing a second ECG signal from the user by the one or more conductive pads when the one or more conductive pads are held by a user's fingers; and sending the second ECG signal to the handheld electrical interface in the control patch.

These and other aspects, their implementations and other features are described in detail in the drawings, the description and the claims.

### BRIEF DESCRIPTION OF THE DRAWINGS

FIG. 1 illustrates a modular ECG recording system capable of conducting ECG recordings in wearable and handheld configurations in accordance with the present invention.

FIGS. 2A and 2B illustrate a wearable configuration of the modular ECG recording system for long-term measurements of ECG signals in accordance with some embodiments of the present invention.

FIGS. 3A and 3B are respectively top view and a cross-sectional view of the wearable configuration of the modular ECG recording system in accordance with some embodiments of the present invention.

FIG. 4 illustrates the handheld configuration of the modular ECG recording system in accordance with some embodiments of the present invention.

FIG. 5 is a system block diagram of a control system in a control patch of the modular ECG recording system in accordance with some embodiments of the present invention.

FIG. 6 is top view of another wearable configuration of the modular ECG recording system in accordance with some embodiments of the present invention.

FIG. 7 is top view of yet another wearable configuration of the modular ECG recording system in accordance with some embodiments of the present invention.

### DETAILED DESCRIPTION OF THE INVENTION

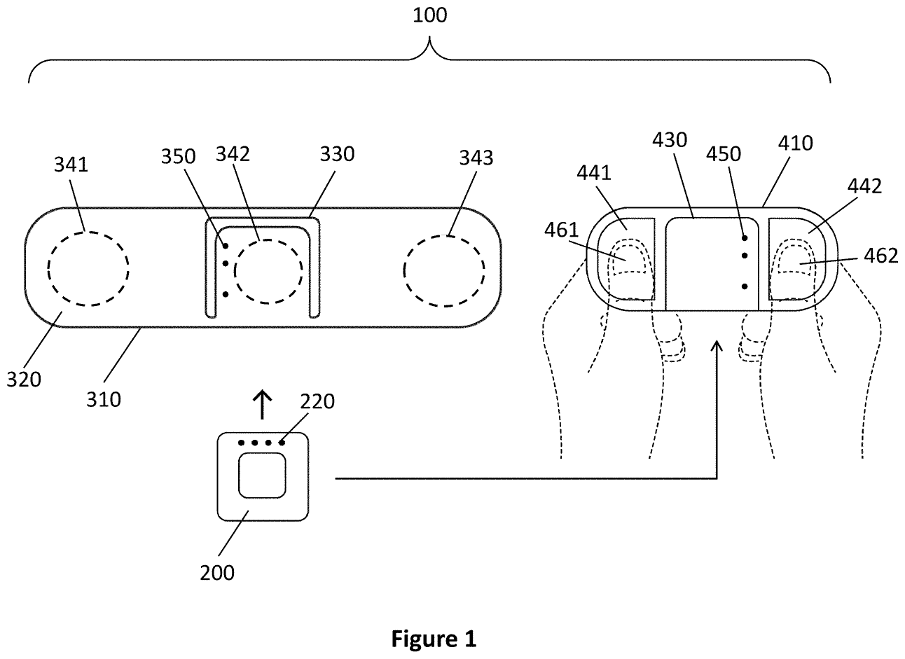

Referring to FIG. 1, a modular ECG recording system 100 includes a control patch 200, a wearable patch 310, and a handheld panel 410. The control patch 200 can be mounted on the wearable patch 310 for ECG recording in a wearable configuration, or mounted on the handheld panel 410 in a handheld configuration. When mounted with the control patch 200, the wearable patch 310 is adapted to be attached to the body or the skin of a user for long-term recording of ECG signals while the user conducts her daily activities. In combination with the control patch 200, the handheld panel 410 can be held by a user's hands for ECG recording. In the present disclosure, the term “wearable patch” can also be referred to as “wearable sticker”, “wearable tag”, or “wearable band”, etc.

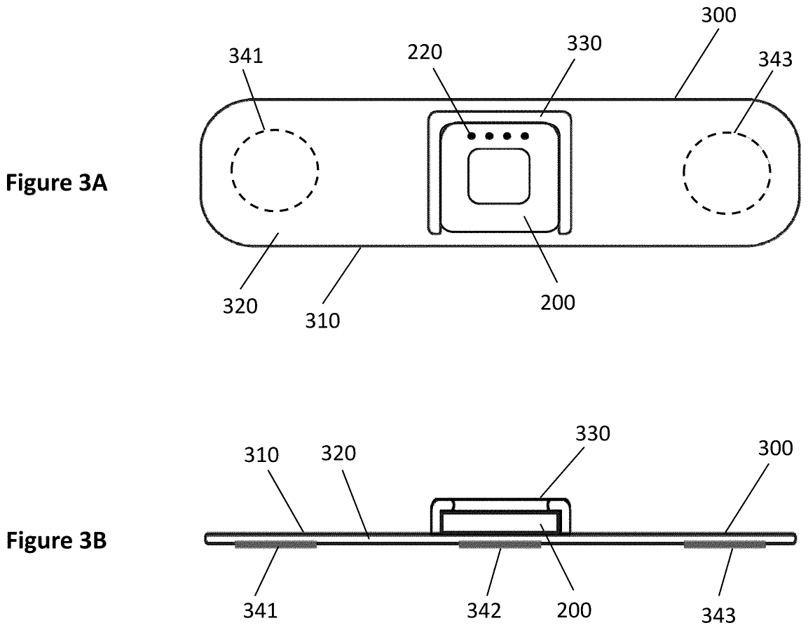

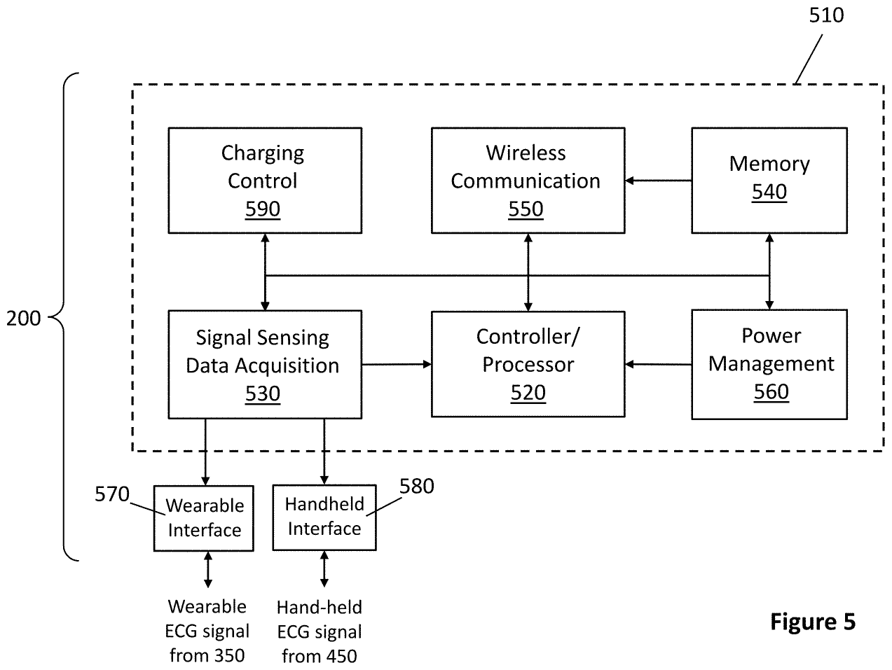

Referring to FIGS. 1-3B, the wearable patch 310 includes a flexible substrate 320, a port 330 configured to receive the control patch 200, sensing electrodes 341-343, and interface electrodes 350. The flexible substrate 320 can be made of soft foam materials such as EVA, PE, CR, PORON, EPD, SCF or fabric textile to provide stretchability and breathability. The port 330 can include a bracket or a recess that can physically secure the control patch 200 to the wearable patch 310. The sensing electrodes 341-343 are in electrical communication with the interface electrodes 350. The lower surface of the flexible substrate 310 can include an adhesive material such as Acrylate or skin hydrogel to attach the wearable patch 310 to the skin of a user 210. When the wearable patch 310 is worn by the user 210, the sensing electrodes 341-343 are configured to sense EEG signals from the body of the user 210. When the control patch 200 is inserted and mounted into the port 330 (FIGS. 3A, 3B), the wearable patch 310 and the control patch 200 together form a wearable configuration 300 of the modular ECG recording system 100. The interface electrodes 350 are electrically connected to a wearable electrical interface (570, FIG. 5) in a control system (510, FIG. 5), which allows the recorded ECG signals to be sent from the sensing electrodes 341-343 to the wearable electrical interface (570, FIG. 5), via interface electrodes 350. Moreover, the one or more sensing electrodes 341-343 can acquire the first ECG signal in the user's body under the control of a controller/processor (520, FIG. 5) in the control patch 200.

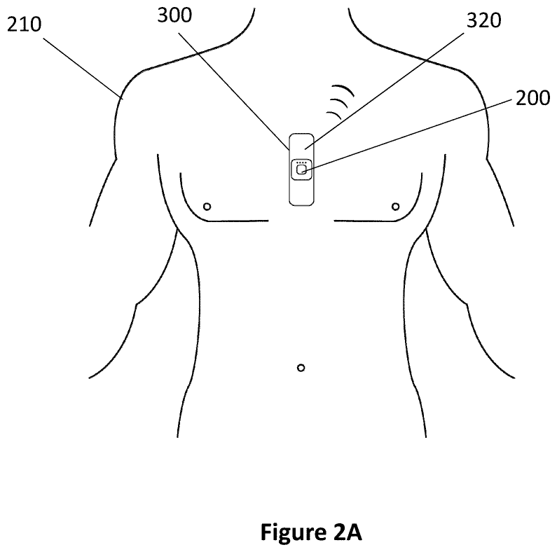

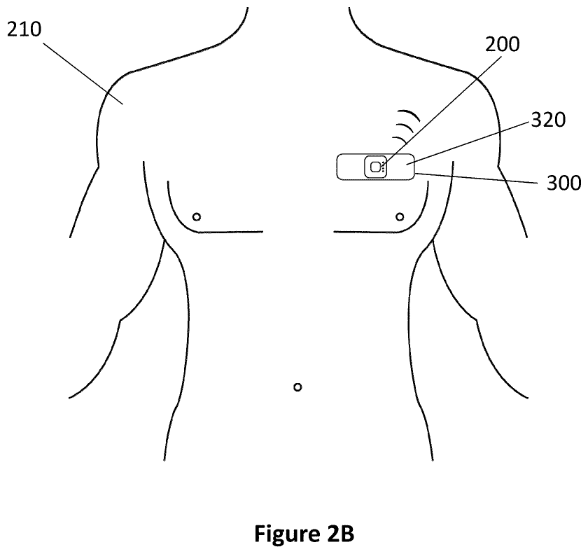

In some embodiments, the sensing electrodes 341-343 can be positioned in a linear array as shown in FIGS. 1 and 3B. The sensing electrodes 342 can also serve as ground for removing noise. Referring to FIGS. 2A and 2B, the wearable configuration 300 of the modular ECG recording system 100 can be attached to the chest of the user 210 for recording ECG signals for a long period of time. The positions and the orientations of the wearable patch 310 can be selected for specific measurement needs. In an example as shown in FIG. 2A, the wearable configuration 300 is positioned at the middle of the chest in a vertical orientation such that the sensing electrodes 341-343 are aligned vertically relative to the user's body. In another example as shown in FIG. 2B, the wearable configuration 300 is positioned on one side of the chest in a horizontal orientation such that the sensing electrodes 341-343 are aligned horizontal relative to the user's body. Multiple wearable configurations 300 can be attached to the center, the left and the right sides of the chest, and other locations on a user for ECG recording in parallel.

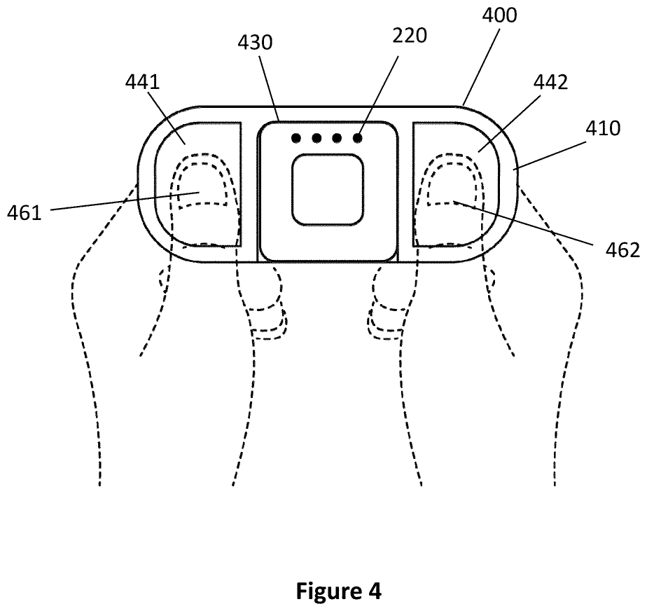

Referring to FIGS. 1 and 4, the handheld panel 410 includes a port 430 and sensing pads 441-442, and interface electrodes 450. The sensing pads 441-442 are in electrical communication with the interface electrodes 450. The handheld panel 410 can be held by a user and allow the user's fingers 461-462 to respectively press against the sensing pads 441-442 for recording ECG signals from the user's body. The port 430 can include a bracket or a recess that can physically secure the control patch 200 to the handheld panel 410. When the control patch 200 is inserted and mounted into the port 430, the handheld panel 410 and the control patch 200 together form a handheld configuration 400 of the modular ECG recording system 100. The interface electrodes 450 are electrically connected to a handheld electrical interface (580, FIG. 5) in a control system (510, FIG. 5), which allows the recorded ECG signals to be sent from the sensing pads 441-442 to the handheld electrical interface (580, FIG. 5), via the interface electrodes 450. Moreover, the sensing pads 441-442 can acquire the first ECG signal in the user's body under the control of a controller/processor (520, FIG. 5) in the control patch 200. A typical ECG recording time for the handheld configuration is between 30 sec and 120 seconds.

In some embodiments, the handheld panel 410 is a portable device powered by internal batteries, which can be conveniently carried by a user. The handheld panel 410 can also be stationary and be connected to line power for an extended period, which, as described below, allows charging of the control patch 200 when it is mounted to the handheld panel 410.

Referring to FIG. 5, a control system 510 in the control patch 200 includes a controller/processor 520, a signal sensing and data acquisition unit 530, a memory 540, a wireless communication unit 550, and a power management unit 560. The signal sensing and data acquisition unit 530 is connected to a wearable interface 570 and a handheld interface 580. The power management unit 560 can include rechargeable batteries for supplying power to various components in the control system 510 and to the charging circuit for the internal batteries, and the control patch 200 (described below).

Referring to FIGS. 1, 3A-3B, 5, in the wearable configuration 300 of the modular ECG recording system 100, the wearable interface 570 is electrically connected to the interface electrodes 350 in the wearable patch 310. ECG signals from long-term recording by the wearable patch 310 are sent to the signal sensing and data acquisition unit 530 via the wearable interface 570. The control patch 200 includes user interface control features 220 such as buttons for a user to control the ECG recording in the wearable configuration 300.

Referring to FIGS. 1, 4-5, in the handheld configuration 400 of the modular ECG recording system 100, the handheld interface 580 is electrically connected to the interface electrodes 450 in the handheld panel 410. ECG signals sensed by the handheld panel 410 are sent to the signal sensing and data acquisition unit 530 via the handheld interface 580. The user interface control features 220 can be used by a user to control the ECG recording in the handheld configuration 400.

The controller/processor 520 can vary parameters of the ECG recordings by the wearable patch 310 and the handheld panel 410. Such measurement parameters can include types, timing, frequencies, durations of measurements, coordination between different wearable patches. The measurement data obtained are stored in the memory 540.

ECG signals acquired are digitized by the controller/processor 520 using a high-resolution ADC circuit, and then stored in the memory 540. The wireless communication unit 550 includes an antenna and power amplifiers. The ECG measurement data can be transmitted wirelessly by the wireless communication unit 550. The wireless communications can be conducted using Wi-Fi, Bluetooth, Near Field Communication (NFC), and other wireless technologies. The ECG measurement data can be sent to a central server and timely reported to a physician, a nurse, or a technician, who can review and decide on further analysis and recommended actions to the user (patient).

An advantageous feature of the disclosed modular ECG recording system is that ECG signals obtained in the wearable configuration and the handheld configuration provide a higher chance of catching arrhythmia or other heart conditions that would otherwise not be possible. It is known that wearing an adhesive for longer period can cause irritation and redness on the skin therefore preventing the user from wearing for multiple times or longer period. During the break period, the handheld configuration allows the user to continue to monitor their heart until the skin heals.

Other advantages of the disclosed modular ECG recording system is that it provides smart options for ECG recordings with adequate retrospective data, convenient and continuous ambulatory recording, extended recording time, and prompt data sharing and analysis.

Furthermore, a wearable patch in the disclosed modular ECG recording system can be disposable, while a control patch can be re-used for a long time, which reduces the cost and complexity of the wearable configuration.

In some embodiments, still referring to FIGS. 1, 4-5, the handheld panel 410 can provide charging to the control patch 200. The control system 510 can include a charging control unit 590 that can charge the rechargeable batteries in the power management unit 560 when the control patch 200 is mounted on the handheld panel 410. The handheld panel 410 can receive power from an external source such as line power or a back-up battery. A user can use the user interface control features 220 to initiate, control, and end the charging process. The back-up battery serves as an ease to charge the control patch on the go in case the user is not near a line power and needs to charge the control patch. The handheld configuration also allows the user to measure their ECG on-demand, which provides great signal quality and fidelity due to the long separation between the two hands.

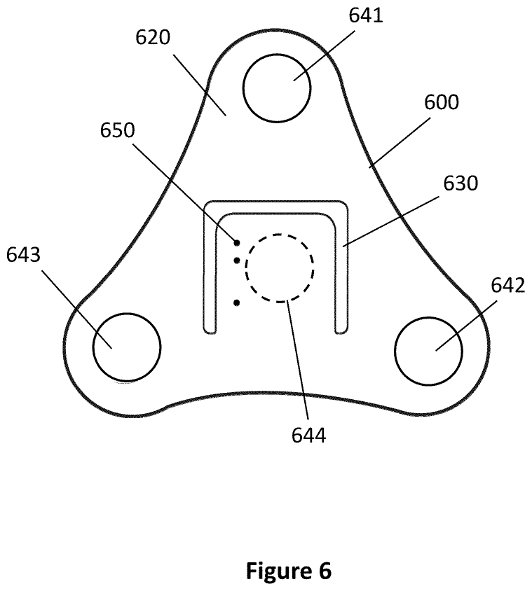

In some embodiments, the wearable patch compatible with the disclosed modular ECG recording system can include different shapes and different number of sensing electrodes. Referring to FIG. 6, a wearable patch 600 includes a flexible substrate 620, three sensing electrodes 641-644, and a port 630 for housing the control patch 200 (FIGS. 1-3B). One of the sensing electrodes 641-644 can serve as ground for removing noise. The three sensing electrodes 641-644 can capture ECG signals in 6 lead configurations. The wearable patch 600 on a user's body can be similar to those shown in FIGS. 2A and 2B.

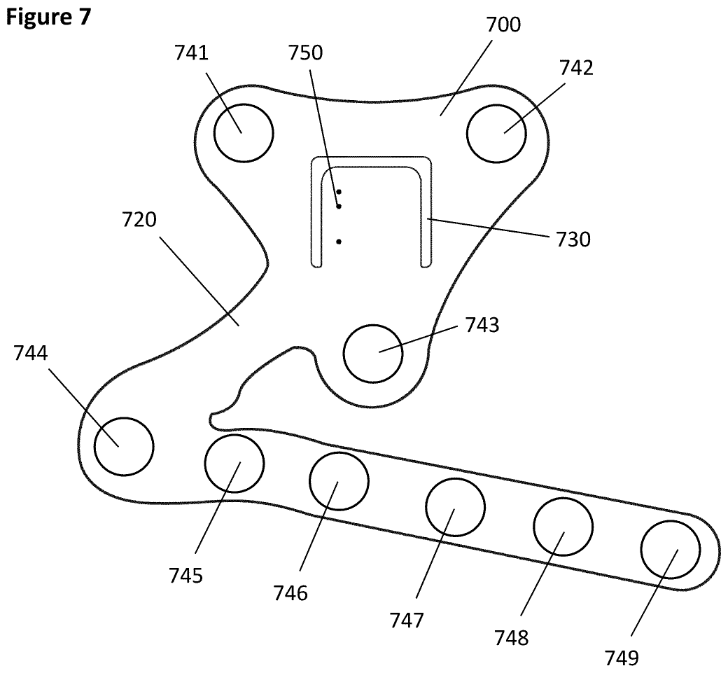

In some embodiments, FIG. 7 shows a wearable patch 700 includes a flexible substrate 720, three sensing electrodes 741-749 along with a ground electrode (not shown), and a port 730 for housing the control patch 200 (FIGS. 1-3B). One of the sensing electrodes 741-749 can serve as ground for removing noise. The sensing electrodes 741-749 can capture ECG signals in 12 lead configurations. The wearable patch 600 on a user's body can be similar to those shown in FIGS. 2A and 2B.

Other details about wearable patches capable of performing measurement and charging functions are disclosed in commonly assigned U.S. patent application Ser. No. 15/423,585, titled “A wearable patch comprising three electrodes for measurement and charging”, filed Feb. 3, 2017, commonly assigned U.S. patent application Ser. No. 15/457,532 (issued as U.S. Pat. No. 10,111,618), titled “Dual purpose wearable patch for measurement and treatment”, filed Mar. 13, 2017, commonly assigned U.S. patent application Ser. No. 15/472,641, titled “Multi-purpose wearable patch for measurement and treatment”, filed Mar. 29, 2017, and commonly assigned U.S. patent application Ser. No. 15/880,445, titled “A smart telehealth ECG recording system”, filed Jan. 25, 2018. The disclosures in the above applications are incorporated herein by reference.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A modular electrocardiogram recording system, comprising:
a control patch comprising:
a control system;
a wearable electrical interface; and
a handheld interface;
a wearable patch configured to be electrically connected to the control patch and to be worn on a user's skin, the wearable patch comprising:
a flexible substrate;
one or more sensing electrodes configured to be in contact with the user's skin to sense a first ECG signal; and
first interface electrodes in electrical communications with the one or more sensing electrodes,
wherein the first interface electrodes are configured to electrically connect to the wearable electrical interface in the control patch and to send the first ECG signal to the wearable electrical interface; and
a handheld panel configured to be electrically connected to the control patch and to be hand held by the user, the handheld panel comprising:
one or more conductive pads configured to be held by and to be in contact with a user's fingers; and
second interface electrodes in electrical communications with the one or more conductive pads,
wherein the one or more conductive pads are configured to sense a second ECG signal from the user, wherein the second interface electrodes are configured to electrically connect to the handheld electrical interface in the control patch and to send the second ECG signal to the handheld electrical interface.

2. The modular electrocardiogram recording system of claim 1, wherein the wearable patch comprises a first port configured to receive the control patch, which causes the first interface electrodes to electrically connect to the wearable electrical interface in the control patch.

3. The modular electrocardiogram recording system of claim 2, wherein the first port includes a bracket or a recess configured to secure the control patch to the wearable patch.

4. The modular electrocardiogram recording system of claim 1, wherein the wearable patch includes an adhesive material to enable the wearable patch to adhere to the user's skin, which is configured to attach the sensing electrodes to be in contact with the user's skin.

5. The modular electrocardiogram recording system of claim 1, wherein the handheld panel comprises a second port configured to receive the control patch, which causes the second interface electrodes to electrically connect to the handheld electrical interface in the control patch.

6. The modular electrocardiogram recording system of claim 5, wherein the second port includes a bracket or a recess configured to secure the control patch to the handheld panel.

7. The modular electrocardiogram recording system of claim 1, wherein the control patch includes a signal sensing and data acquisition unit in communication with the wearable electrical interface and the handheld interface, wherein the signal sensing and data acquisition unit is configured to receive the first ECG signal via the wearable electrical interface and configured to receive the second ECG signal via the handheld electrical interface.

8. The modular electrocardiogram recording system of claim 7, wherein the control system further comprises a wireless communication unit configured to transmit ECG data to an external device, the ECG data being based on at least one of the first ECG signal and the second ECG signal.

9. The modular electrocardiogram recording system of claim 7, wherein the control system further comprises a controller/processor configured to analyze the first ECG signal and the second ECG signal to produce the ECG data.

10. The modular electrocardiogram recording system of claim 9, wherein the one or more sensing electrodes in the wearable patch are configured to sense the first ECG signal in the user's body under the control of the controller/processor.

11. The modular electrocardiogram recording system of claim 9, wherein the one or more conductive pads in the handheld panel are configured to sense the second ECG signal in the user's body under the control of the controller/processor.

12. The modular electrocardiogram recording system of claim 7, wherein the control system further comprises rechargeable batteries and a charging unit, wherein the charging unit is configured to charge the rechargeable batteries when the control patch is mounted on the handheld panel, wherein the handheld panel is configured to receive power from an external source or a back-up battery.

13. The modular electrocardiogram recording system of claim 1, wherein one of the one or more sensing electrodes is used as grounding.

14. A method for configurable electrocardiogram recordings, comprising:
mounting a control patch to a wearable patch to be worn on a user's skin,
wherein the control patch includes a control system, a wearable electrical interface, and a handheld interface, wherein the wearable patch includes a flexible substrate, one or more sensing electrodes, and first interface electrodes in electrical communications with the one or more sensing electrodes;
electrically connecting the first interface electrodes to the wearable electrical interface in the control patch;
sensing a first ECG signal from the user when the wearable patch is in contact with the user's skin;
sending the first ECG signal to the wearable electrical interface in the control patch;
mounting the control patch to a handheld panel comprising one or more conductive pads configured to be held by a user's fingers and second interface electrodes in electrical communications with the one or more conductive pads;
electrically connecting the second interface electrodes to the handheld electrical interface in the control patch;
sensing a second ECG signal from the user by the one or more conductive pads when the one or more conductive pads are held by a user's fingers; and
sending the second ECG signal to the handheld electrical interface in the control patch.

15. The method of claim 14, further comprising:
transmitting, from the control patch to an external device, ECG data based on at least one of the first ECG signal and the second ECG signal.

16. The method of claim 15, wherein the ECG data is produced based on the first ECG signal and the second ECG signal.

17. The method of claim 14, further comprising:
sensing the first ECG signal in the user's body by the one or more sensing electrodes in the wearable patch under control of a controller/processor in the control patch.

18. The method of claim 14, further comprising:
sensing the second ECG signal in the user's body by the one or more conductive pads in the handheld panel under control of a controller/processor in the control patch.

19. The method of claim 14, further comprising:
charging rechargeable batteries in the control patch when the control patch is mounted on the handheld panel.
